(19) United States
(12) Patent Application Publication
(10) Pub. No.: US 2026/0294404 A1
(43) Pub. Date: Oct. 1, 2026

(54) ULTRASOUND PROBE, ULTRASOUND SYSTEM, AND CONTROL METHOD OF ULTRASOUND PROBE

(71) Applicant: FUJIFILM Corporation, Tokyo (JP)

(72) Inventor: Hiroyuki KARASAWA, Kanagawa (JP)

(73) Assignee: FUJIFILM Corporation, Tokyo (JP)

(21) Appl. No.: 19/565,543

(22) Filed: Mar. 13, 2026

(30) Foreign Application Priority Data
Mar. 31, 2025 (JP) 2025-058404

(51) Int. Cl. A61B 8/00

(52) U.S. Cl. CPC A61B 8/56; A61B 8/4433; A61B 8/4472

(57) ABSTRACT

Provided are an ultrasound probe, an ultrasound system, and a control method of an ultrasound probe, in which a sufficient remaining level of a built-in battery can be ensured in an ultrasound examination while a life of the built-in battery of the ultrasound probe is maintained for a long time. An ultrasound probe, which is wirelessly connected to an ultrasound apparatus main body, includes a transducer array, a built-in battery, a power supply controller that controls an on state and an off state of a power supply of the ultrasound probe, and a charging controller that charges the built-in battery by using power supplied from an outside of the ultrasound probe, in which the charging controller charges the built-in battery at charging levels different depending on the on state and the off state of the power supply.

### CROSS-REFERENCE TO RELATED APPLICATIONS

The present application claims priority under 35 U.S.C. § 119 to Japanese Patent Application No. 2025-058404, filed on March 31, 2025. The above application is hereby expressly incorporated by reference, in its entirety, into the present application.

### BACKGROUND OF THE INVENTION

### 1. Field of the Invention

The present invention relates to an ultrasound probe including a built-in battery, an ultrasound system including the ultrasound probe including the built-in battery, and a control method of the ultrasound probe including the built-in battery.

### 2. Description of the Related Art

In the related art, an ultrasound examination has been performed in which an ultrasound image representing a tomographic plane inside a subject is captured using a so-called ultrasound probe, and the inside of the subject is examined based on the captured ultrasound image. In order to smoothly perform such an ultrasound examination, for example, as disclosed in JP2013-135557A, an ultrasound probe that incorporates a battery has been developed.

### SUMMARY OF THE INVENTION

In general, it is known that, in a case where charging of the built-in battery of the ultrasound probe up to a charging capacity unique to the built-in battery, that is, charging up to full charge is repeated, a life of the built-in battery of the ultrasound probe is shortened, that is, a chargeable capacity of the built-in battery is reduced, and, on the contrary, in a case where charging is stopped before the built-in battery is fully charged, the life of the built-in battery tends to be maintained for a long time even in a case where the built-in battery is repeatedly charged. However, in a case where the built-in battery is not sufficiently charged in order to maintain the life of the built-in battery for a long time, there is a concern that a remaining level of the built-in battery may be insufficient during the ultrasound examination.

The present invention has been made to solve such a problem in the related art, and an object of the present invention is to provide an ultrasound probe, an ultrasound system, and a control method of an ultrasound probe, in which a sufficient remaining level of a built-in battery can be ensured in an ultrasound examination while a life of the built-in battery of the ultrasound probe is maintained for a long time.

The above-described object can be achieved with the following configurations.

1 An ultrasound probe that is wirelessly connected to an ultrasound apparatus main body, the ultrasound probe including:

a transducer array;

a built-in battery;

a power supply controller that controls an on state and an off state of a power supply of the ultrasound probe; and

a charging controller that charges the built-in battery by using power supplied from an outside of the ultrasound probe,

in which the charging controller charges the built-in battery at charging levels different depending on the on state and the off state of the power supply.

2 The ultrasound probe according to 1, in which the charging controller charges the built-in battery at a predetermined first charging level in the off state of the power supply, and charges the built-in battery at a charging level lower than the first charging level in the on state of the power supply.

3 The ultrasound probe according to 2, further including:

an image acquisition unit that acquires an ultrasound image by transmitting and receiving ultrasound waves using the transducer array; and

a wireless communication circuit that wirelessly transmits the ultrasound image acquired by the image acquisition unit to the ultrasound apparatus main body,

in which the charging controller charges the built-in battery at charging levels different depending on whether the ultrasound probe is in a connection state of being wirelessly connected to the ultrasound apparatus main body via the wireless communication circuit or in a non-connection state of being not wirelessly connected to the ultrasound apparatus main body.

4 The ultrasound probe according to 3, in which the charging controller charges the built-in battery at a predetermined second charging level in the non-connection state, and charges the built-in battery at a third charging level lower than the second charging level in the connection state.

5 The ultrasound probe according to any one of 1 to 4, further including:

a power receive coil that is connected to the charging controller,

in which the charging controller charges the built-in battery by using the power wirelessly supplied from the outside of the ultrasound probe via the power receive coil.

6 An ultrasound system including:

the ultrasound probe according to any one of 1 to 5; and

the ultrasound apparatus main body that is wirelessly connected to the ultrasound probe.

7 A control method of an ultrasound probe including a transducer array, a built-in battery, a power supply controller that controls an on state and an off state of a power supply of the ultrasound probe, and a charging controller that charges the built-in battery by using power supplied from an outside of the ultrasound probe, the control method including:

charging the built-in battery at charging levels different depending on the on state and the off state of the power supply.

In the present invention, the ultrasound probe that is wirelessly connected to an ultrasound apparatus main body includes the transducer array, the built-in battery, the power supply controller that controls the on state and the off state of the power supply of the ultrasound probe, and the charging controller that charges the built-in battery by using the power supplied from the outside of the ultrasound probe, and the charging controller charges the built-in battery at charging levels different depending on the on state and the off state of the power supply, so that the sufficient remaining level of the built-in battery can be secured in the ultrasound examination while the life of the built-in battery of the ultrasound probe is maintained for a long time.

### BRIEF DESCRIPTION OF THE DRAWINGS

FIG. 1 is a block diagram showing a configuration of an ultrasound system including an ultrasound probe according to an embodiment of the present invention and an ultrasound apparatus main body connected to the ultrasound probe.

FIG. 2 is a block diagram showing an internal configuration of a transmission and reception circuit in the embodiment of the present invention.

FIG. 3 is a block diagram showing an internal configuration of an image generation unit in the embodiment of the present invention.

FIG. 4 is a flowchart showing an operation of a charging controller in the ultrasound probe according to the embodiment of the present invention.

### DESCRIPTION OF THE PREFERRED EMBODIMENTS

Hereinafter, an embodiment of the present invention will be described with reference to the accompanying drawings.

Description of configuration requirements written below is provided based on a representative embodiment of the present invention, but the present invention is not limited to such an embodiment.

In the present specification, numerical ranges represented by “to” include numerical values before and after “to” as lower limit values and upper limit values.

In the present specification, “the same” includes an error range generally allowed in the technical field.

### Embodiment

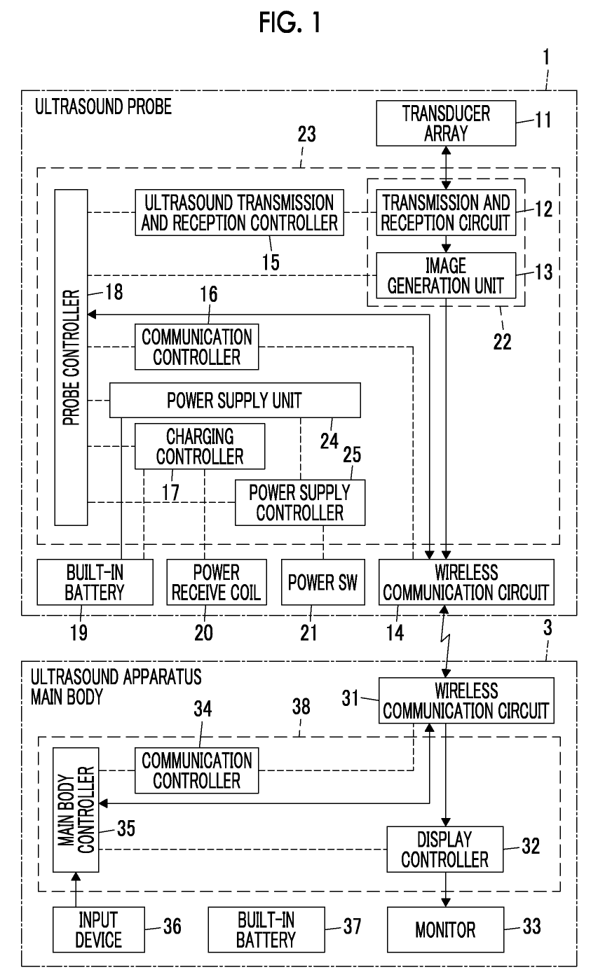

FIG. 1 shows a configuration of an ultrasound system according to the embodiment of the present invention. The ultrasound system includes an ultrasound probe 1 according to the embodiment of the present invention and an ultrasound apparatus main body 3 that is wirelessly connected to the ultrasound probe 1.

The ultrasound probe 1 includes a transducer array 11, and a transmission and reception circuit 12, an image generation unit 13, and a wireless communication circuit 14 are sequentially connected to the transducer array 11. In addition, an ultrasound transmission and reception controller 15 is connected to the transmission and reception circuit 12. A communication controller 16 is connected to the wireless communication circuit 14. In addition, the ultrasound probe 1 includes a charging controller 17. A built-in battery 19 and a power receive coil 20 are connected to the charging controller 17. A power supply unit 24 is connected to the built-in battery 19. A power supply controller 25 is connected to the power supply unit 24. A power switch (SW) 21 is connected to the power supply controller 25. In addition, a probe controller 18 is connected to the image generation unit 13, the wireless communication circuit 14, the ultrasound transmission and reception controller 15, the communication controller 16, the charging controller 17, the power supply unit 24, and the power supply controller 25.

The transmission and reception circuit 12 and the image generation unit 13 constitute an image acquisition unit 22. In addition, a probe processor 23 for the ultrasound probe 1 is configured by the transmission and reception circuit 12, the image generation unit 13, the ultrasound transmission and reception controller 15, the communication controller 16, the charging controller 17, the probe controller 18, the power supply unit 24, and the power supply controller 25.

The ultrasound apparatus main body 3 includes a wireless communication circuit 31, and a display controller 32 and a monitor 33 are sequentially connected to the wireless communication circuit 31. A communication controller 34 is connected to the wireless communication circuit 31. In addition, a main body controller 35 is connected to the wireless communication circuit 31, the display controller 32, and the communication controller 34. An input device 36 is connected to the main body controller 35. In addition, the ultrasound apparatus main body 3 includes a built-in battery 37. In addition, a main body processor 38 for the ultrasound apparatus main body 3 is configured by the display controller 32, the communication controller 34, and the main body controller 35.

The transducer array 11 of the ultrasound probe 1 has a plurality of ultrasound oscillators that are one-dimensionally or two-dimensionally arranged. In accordance with a drive signal supplied from the transmission and reception circuit 12, each of the ultrasound oscillators transmits ultrasound and receives an ultrasound echo from a subject to output a signal based on the ultrasound echo. Each ultrasound oscillator is configured by forming an electrode at both ends of a piezoelectric body consisting of, for example, a piezoelectric ceramic represented by lead zirconate titanate (PZT), a polymer piezoelectric element represented by polyvinylidene difluoride (PVDF), and a piezoelectric single crystal represented by a lead magnesium niobate-lead titanate (PMN-PT).

The image acquisition unit 22 configured by the transmission and reception circuit 12 and the image generation unit 13 acquires an ultrasound image in which a tomographic plane inside the subject is imaged by transmitting and receiving an ultrasound beam using the ultrasound probe 1.

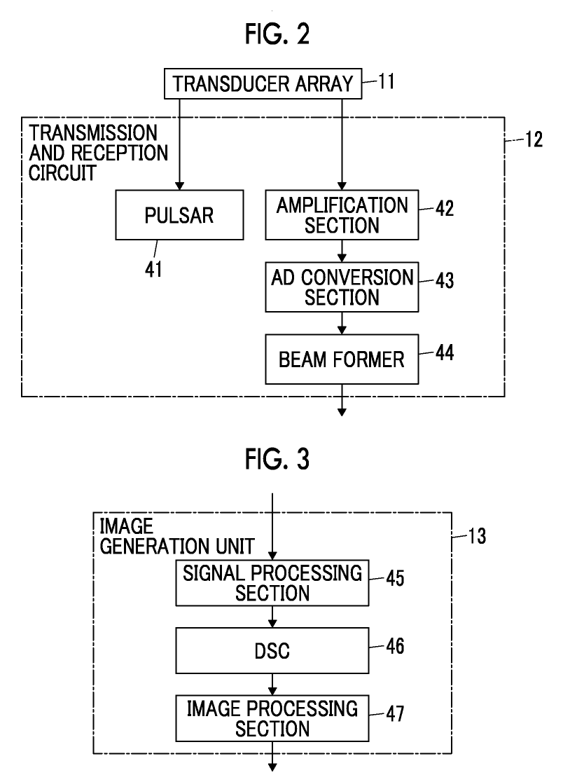

The transmission and reception circuit 12 causes the transducer array 11 to transmit the ultrasonic wave and generates a sound ray signal on the basis of a reception signal acquired by the transducer array 11, under the control of the probe controller 18. As shown in FIG. 2, the transmission and reception circuit 12 includes a pulser 41 connected to the transducer array 11, and an amplification section 42, an analog-to-digital (AD) conversion section 43, and a beam former 44 that are sequentially connected in series to the transducer array 11.

The pulser 41 includes, for example, a plurality of pulse generators, and supplies each drive signal to the plurality of ultrasound oscillators of the transducer array 11 such that the ultrasound waves transmitted from the plurality of ultrasound oscillators of the transducer array 11 form an ultrasound beam, by adjusting a delay amount based on a transmission delay pattern selected in response to a control signal from the ultrasound transmission and reception controller 15 and the probe controller 18. In this way, in a case where a pulsed or continuous wave-like voltage is applied to the electrodes of the ultrasound oscillator of the transducer array 11, the piezoelectric body expands and contracts to generate a pulsed or continuous wave-like ultrasound wave from each of the ultrasound oscillators, thereby forming an ultrasound beam from the combined wave of these ultrasound waves.

The transmitted ultrasound beam is reflected in a target, for example, a site of the subject, and propagates toward the transducer array 11 of the ultrasound probe 1. The ultrasound echo propagating toward the transducer array 11 is received by each ultrasound oscillator constituting the transducer array 11. In this case, each of the ultrasound oscillators constituting the transducer array 11 receives the propagating ultrasound echo to expand and contract to generate a reception signal, which is an electrical signal, and outputs these reception signals to the amplification section 42.

The amplification section 42 amplifies the signal input from each of the ultrasound oscillators constituting the transducer array 11 and transmits the amplified signal to the AD conversion section 43. The AD conversion section 43 converts the signal transmitted from the amplification section 42 into digital reception data. The beam former 44 performs so-called reception focus processing by applying and adding the delay to each reception data received from the AD conversion section 43. By the reception focus processing, each reception data, which is converted by the AD conversion section 43, is phase-added, and the sound ray signal in which the focus of the ultrasound echo is narrowed down is acquired.

As shown in FIG. 3, the image generation unit 13 includes a signal processing section 45, a digital scan converter (DSC) 46, and an image processing section 47 that are connected in series in order.

The signal processing section 45 generates a B-mode image signal, which is tomographic image information regarding tissues inside the subject, by performing, on the sound ray signal received from the transmission and reception circuit 12, correction of the attenuation due to the distance according to the depth of the reflection position of the ultrasound wave using a sound velocity value set by the probe controller 18 and then performing envelope detection processing.

The DSC 46 converts (raster-converts) the B-mode image signal generated by the signal processing section 45 into an image signal in accordance with a normal television signal scanning method.

The image processing section 47 performs various necessary image processing such as gradation processing on the B-mode image signal input from the DSC 46, and then transmits the B-mode image signal to the wireless communication circuit 14. Hereinafter, the B-mode image signal that has been subjected to image processing by the image processing section 47 is referred to as an ultrasound image.

The ultrasound transmission and reception controller 15 controls the transmission and reception circuit 12 to transmit and receive the ultrasound waves according to transmission/reception conditions determined in the transducer array 11. The determined transmission/reception conditions may include, for example, a transmission pattern of the ultrasound waves, a transmission focus position of the ultrasound waves, a reception focus position, a display depth of the ultrasound image, and conditions of a gain and a dynamic range of the reception signal.

The wireless communication circuit 14 is a circuit that wirelessly transmits the ultrasound image and the like acquired by the image acquisition unit 22 to the ultrasound apparatus main body 3, and wirelessly transmits and receives other necessary information to and from the ultrasound apparatus main body 3.

The communication controller 16 controls the transmission and reception of information to and from the ultrasound apparatus main body 3 via the wireless communication circuit 14. The communication controller 16 can cause the wireless communication circuit 14 to transmit and receive, for example, information for the ultrasound probe 1 to wirelessly connect to the ultrasound apparatus main body 3, and transmit the ultrasound image to the ultrasound apparatus main body 3.

The power supply unit 24 converts the power supplied from the built-in battery 19 into a voltage suitable for each unit in the ultrasound probe 1, such as the transducer array 11, the transmission and reception circuit 12, the image generation unit 13, the wireless communication circuit 14, the ultrasound transmission and reception controller 15, and the communication controller 16, and supplies the converted power.

The power switch (power SW) 21 is a switch for turning on or off the power supply unit 24. The power switch 21 is operated by, for example, a user. Information representing an instruction to turn on the power supply unit 24 and information representing an instruction to turn off the power supply unit 24, which are input via the power switch 21, are transmitted to the power supply controller 25.

The power supply controller 25 controls the on state and the off state of the power supply unit 24. The power supply controller 25 turns on or off the power supply unit 24 based on, for example, information input by the power switch 21. Hereinafter, the on state of the power supply unit 24 is referred to as the on state of the power supply of the ultrasound probe 1, and the off state of the power supply unit 24 is referred to as the off state of the power supply of the ultrasound probe 1.

The power receive coil 20 receives the power supplied from the wireless charger by sensing a magnetic field emitted from an external wireless charger. In addition, the power receive coil 20 emits a response signal in response to a search signal transmitted from the wireless charger.

The built-in battery 19 of the ultrasound probe 1 supplies power to the power supply unit 24. The built-in battery 19 is charged by receiving power from an external wireless charger via the power receive coil 20, for example. As the built-in battery 19, various batteries can be used, but for example, a so-called lithium ion battery can be used.

By the way, the higher the voltage used for charging, the faster and the larger the capacity of the built-in battery 19 can be charged. On the other hand, in a case where the built-in battery 19 is repeatedly charged up to the charging capacity unique to the built-in battery 19, that is, up to full charge, by a high charging voltage, the life of the built-in battery 19 may be shortened, that is, the capacity that can be charged at once may be reduced. Examples of a situation in which the built-in battery 19 is charged a plurality of times in a short period of time include a situation in which a state in which the built-in battery 19 is charged with the power supply of the ultrasound probe 1 turned on during the ultrasound examination is alternately repeated with a state in which the ultrasound probe 1 is used with the charging released.

In addition, in a case where the power supply of the ultrasound probe 1 is turned on, the ultrasound probe 1 may be heated by driving. Further, the ultrasound probe 1 may be heated by the rapid charging of the built-in battery 19, and the built-in battery 19 may be further heated in a case where the power supply of the ultrasound probe 1 is turned on and the built-in battery 19 supplies a large amount of power. Therefore, for example, in a case where the built-in battery 19 is rapidly charged in a case where the power supply of the ultrasound probe 1 is turned on, the temperature of the built-in battery 19 is likely to increase due to three elements of driving of the ultrasound probe 1, rapid charging, and a combination thereof. It is known that maintaining the built-in battery 19 at, for example, a high temperature of 40°C or higher is one of the factors that shorten the life of the built-in battery 19.

In addition, examples of a situation in which the built-in battery 19 is sufficiently time-stably maintained in a charged state include a situation in which the ultrasound examination of the subject is not performed. In a case where the ultrasound examination of the subject is not performed, the power supply of the ultrasound probe 1 is often turned off in order to suppress the power consumption of the built-in battery 19.

Therefore, the charging controller 17 charges the built-in battery 19 by using the power supplied from the outside of the ultrasound probe 1 via the power receive coil 20, and in this case, charges the built-in battery 19 at different charging levels according to the on state and the off state of the power supply of the ultrasound probe 1. The charging controller 17 can charge the built-in battery 19 at a first charging level determined in the off state of the power supply of the ultrasound probe 1, and can charge the built-in battery 19 at a charging level lower than the first charging level in the on state of the power supply of the ultrasound probe 1, for example. Here, the charging level refers to at least one of a charging voltage or a charging current.

As a result, the number of times of charging at a high charging level can be reduced, the life of the built-in battery 19 of the ultrasound probe 1 can be maintained long, and a sufficient remaining level of the built-in battery 19 can be ensured in the ultrasound examination. In addition, since the built-in battery 19 is charged at a charging level lower than the first charging level in the on state of the power supply of the ultrasound probe 1, the adverse effect of the temperature rise of the built-in battery 19 on the life of the built-in battery 19 can be reduced.

In addition, the charging controller 17 can also charge the built-in battery 19 at different charging levels according to a connection state in which the ultrasound probe 1 is wirelessly connected to the ultrasound apparatus main body 3 and a non-connection state in which the ultrasound probe 1 is not wirelessly connected to the ultrasound apparatus main body 3, via the wireless communication circuit 14. The charging controller 17 determines that the ultrasound probe 1 is less likely to be used in the non-connection state than in the connection state, and can charge the built-in battery 19 at a second charging level determined in the non-connection state, and can charge the built-in battery 19 at a third charging level lower than the second charging level in the connection state, for example. In addition, since the power consumption in the ultrasound probe 1 is larger in the connection state than in the non-connection state, the life of the built-in battery 19 can be maintained long by further reducing the charging level. The first charging level in the off state of the power supply of the ultrasound probe 1 can be set to be larger than the second charging level in the connection state.

The wireless communication circuit 31 of the ultrasound apparatus main body 3 is a circuit that wirelessly receives the ultrasound image and the like from the ultrasound probe 1, and wirelessly transmits and receives necessary information such as information for the ultrasound apparatus main body 3 to wirelessly connect to the ultrasound probe 1.

The communication controller 34 controls the transmission and reception of information to and from the ultrasound probe 1 via the wireless communication circuit 31. The communication controller 34 can cause the wireless communication circuit 31 to receive the ultrasound image from the ultrasound probe 1, transmit and receive information for the ultrasound apparatus main body 3 to wirelessly connect to the ultrasound probe 1, and the like.

The display controller 32 performs a predetermined process on the ultrasound image and the like received from the ultrasound probe 1 under the control of the main body controller 35, and displays the ultrasound image and the like on the monitor 33.

The monitor 33 displays the ultrasound image and the like received from the ultrasound probe 1 under the control of the display controller 32, and is, for example, a liquid crystal display (LCD), an organic electroluminescence display (organic EL display), or the like.

The input device 36 is for a user to perform an input operation, and includes, for example, a device such as a keyboard, a mouse, a track ball, a touch pad, and a touch sensor disposed on the monitor 33 in a superimposed manner.

The built-in battery 37 of the ultrasound apparatus main body 3 supplies power to the entire ultrasound apparatus main body 3. The built-in battery 37 is charged by, for example, an external power supply device (not shown) connected to an alternating current power supply C having a specified voltage value and configured by a so-called alternating current adapter (AC adapter) or the like. As the built-in battery 37, various batteries can be used, but for example, a lithium ion battery can be used.

In the present embodiment, each process in the probe processor 23 and the main body processor 38 is executed by any computer. In addition, any computer may execute these processes using a processor as hardware, a program as software, or a combination thereof. In such a case, the processor is configured to execute various types of processing in the present embodiment in cooperation with the program, and may function as each unit or each means in the present embodiment. Additionally, the execution order of the process by the processor is not limited to the order described above and may be changed as appropriate. Any computer may be a general purpose computer, a special purpose computer, a workstation, or other system capable of executing each process.

The probe processor 23 and the main body processor 38 may be configured by one or a plurality of pieces of hardware, and the type of the hardware is not limited. For example, the probe processor 23 and the main body processor 38 may be configured by a central processing unit (CPU), a micro processing unit (MPU), a programmable logic device such as a field programmable gate array (FPGA), a dedicated circuit for executing specific processing such as an application specific integrated circuit (ASIC), a graphic processing unit (GPU), a neural processing unit (NPU), or hardware. Additionally, the type of hardware may be a combination of different types of hardware. In a case where a plurality of hardware components are configured to execute one or a plurality of processes of a certain processor, the plurality of hardware components may be present in physically separate devices or may be present within the same device. In addition, in any embodiment, an order of each process by the probe processor 23 and the main body processor 38 is not limited to the above-described order, and may be appropriately changed. In addition, hardware is implemented in a form of an electric circuit (circuitry) in which circuit elements, such as semiconductor elements, are combined.

Further, the program may be software, such as firmware or a microcode. The program may also be, for example, a group of program modules, and each function may be implemented by a processor configured to execute the corresponding function. The program may be a program code or a plurality of code segments that are stored in one or a plurality of non-transitory computer-readable media (for example, storage media or other storages). The program may be divided and stored in a plurality of non-transitory computer-readable media present in devices physically separated from each other. The program code or the code segment may represent any combination of procedures, functions, subprograms, routines, subroutines, modules, software packages, classes, instructions, data structures, or program statements. The program code or code segments may be connected to other code segments or hardware circuits by transmitting and receiving information, data, arguments, parameters, or contents of a memory.

Next, an example of an appearance of the ultrasound system will be described. The ultrasound system can include a body holder that holds the ultrasound apparatus main body 3, and a holding stand that includes a probe holder that holds the ultrasound probe 1. The probe holder holds the ultrasound probe 1, for example, in a standing state.

The probe holder can include a probe charger that charges the ultrasound probe 1. The probe charger can include, for example, a power transmission coil that generates a magnetic field for power supply to the ultrasound probe 1, and the power transmission coil and the power receive coil 20 of the ultrasound probe 1 are disposed to face each other in a state in which the ultrasound probe 1 is held by the probe holder. The probe charger is electrically connected to the ultrasound apparatus main body 3 via a wiring line disposed in the holding stand, and in a case where the ultrasound probe 1 is held by the probe holder, the probe charger can supply power to the ultrasound probe 1 from the built-in battery 37 of the ultrasound apparatus main body 3 via the probe charger.

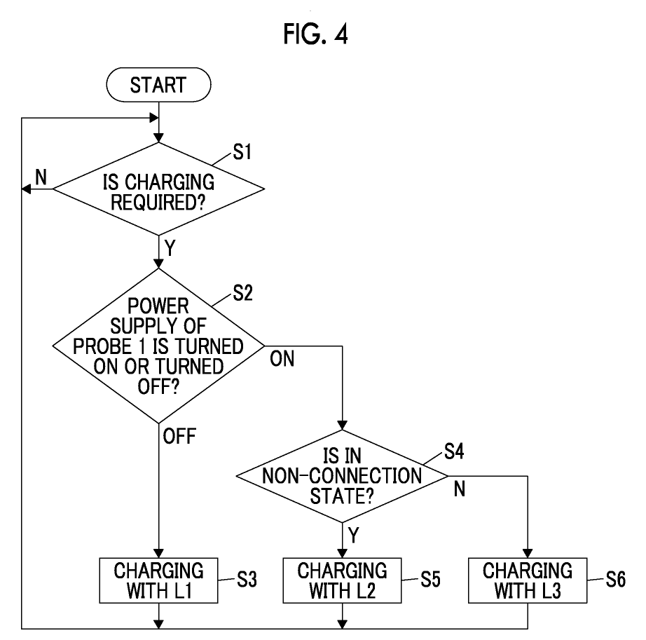

Next, an operation of the charging controller 17 in the ultrasound probe 1 according to the embodiment of the present invention will be described with reference to the flowchart of FIG. 4.

In step S1, the charging controller 17 determines whether or not charging is necessary for the built-in battery 19 of the ultrasound probe 1. The charging controller 17 can determine that charging is necessary in a case where the built-in battery 19 is in a state in which charging is possible, for example, by detecting a connection state between the ultrasound probe 1 and a charger for the ultrasound probe 1. The charging controller 17 can determine that charging is necessary in a case where the ultrasound probe 1 is connected to the charger, for example. In addition, the charging controller 17 can determine that charging is not necessary in a case where the ultrasound probe 1 is not connected to the charger.

The process of step S1 is repeated as long as it is determined in step S1 that charging is not necessary for the built-in battery 19 of the ultrasound probe 1. In a case where it is determined in step S1 that charging is necessary for the built-in battery 19 of the ultrasound probe 1, the process proceeds to step S2.

In step S2, the charging controller 17 determines whether the power supply of the ultrasound probe 1 is turned on or turned off. In a case where it is determined in step S2 that the power supply of the ultrasound probe 1 is turned off, the process proceeds to step S3.

In step S3, the charging controller 17 charges the built-in battery 19 of the ultrasound probe 1 at a first charging level L1. The charging at the first charging level L1 is, for example, charging at a high charging voltage such as 4.2 V, and the built-in battery 19 can be charged to the maximum capacity of the built-in battery 19. In the off state of the power supply of the ultrasound probe 1, the number of times of charging is small because the built-in battery 19 is sufficiently time-stably charged, for example, once a day at night or the like when the user does not use the ultrasound probe 1. In addition, in the off state of the power supply of the ultrasound probe 1, since there is no heat generation due to the driving of the ultrasound probe 1, the temperature of the built-in battery 19 is less likely to rise than in the on state of the power supply of the ultrasound probe 1. Therefore, the built-in battery 19 can be charged to the maximum capacity while the life of the built-in battery 19 is maintained long by step S3.

In a case where it is determined in step S2 that the power supply of the ultrasound probe 1 is turned on, the process proceeds to step S4. In step S4, the charging controller 17 determines whether or not the ultrasound probe 1 is in a non-connection state with respect to the ultrasound apparatus main body 3 by referring to information on the connection state between the ultrasound probe 1 and the ultrasound apparatus main body 3 transmitted and received by the wireless communication circuit 14. In a case where it is determined in step S4 that the ultrasound probe 1 is in a non-connection state with respect to the ultrasound apparatus main body 3, the process proceeds to step S5.

In step S5, the charging controller 17 charges the built-in battery 19 of the ultrasound probe 1 at a second charging level L2, which is lower than the first charging level L1 in step S3, for example, a charging voltage of 4.1 V. In a case where the power supply of the ultrasound probe 1 is turned on but the ultrasound probe 1 is not connected to the ultrasound apparatus main body 3, the ultrasound probe 1 is often used immediately before or after the ultrasound examination of the subject. Therefore, in a case where the ultrasound probe 1 is in the non-connection state, the frequency of charging the built-in battery 19 may be high, for example, as compared with the off state of the power supply of the ultrasound probe 1, and the number of times the remaining amount of the built-in battery 19 reaches the maximum capacity corresponding to the charging level used may be large. In addition, in a case where the power supply of the ultrasound probe 1 is turned on, the power consumption is high as compared with the off state, and the temperature of the built-in battery 19 is likely to be high. By charging the built-in battery 19 of the ultrasound probe 1 at the second charging level L2 lower than the first charging level L1, the life of the built-in battery 19 can be maintained long even in a case where the frequency of charging the built-in battery 19 is high as compared with the off state of the power supply of the ultrasound probe 1.

In a case where it is determined in step S4 that the ultrasound probe 1 is in the connection state with respect to the ultrasound apparatus main body 3, the process proceeds to step S6. In step S6, the charging controller 17 charges the built-in battery 19 of the ultrasound probe 1 at a third charging level L3, which is lower than the second charging level L2 in step S5, for example, a charging voltage of 4.0 V. In a case where the power supply of the ultrasound probe 1 is turned on and the ultrasound probe 1 is connected to the ultrasound apparatus main body 3, the ultrasound probe 1 is often used for the ultrasound examination of the subject. In this case, the built-in battery 19 may be frequently charged so that the remaining amount of the built-in battery 19 during the ultrasound examination is not insufficient, and the number of times the remaining amount of the built-in battery 19 reaches the maximum capacity corresponding to the charging level used may be large. In addition, since the ultrasound probe 1 is used for the ultrasound examination, the power consumption is high as compared with a case where the ultrasound probe 1 is in the non-connection state with respect to the ultrasound apparatus main body 3, and the temperature of the built-in battery 19 is likely to be high. By charging the built-in battery 19 at the third charging level L3 lower than the second charging level L2 in step S5, the life of the built-in battery 19 can be maintained long even in a case where the frequency of charging the built-in battery 19 is high.

By the operation of the charging controller 17 described above, the number of times of charging at a high charging level can be reduced, the life of the built-in battery 19 of the ultrasound probe 1 can be maintained long, and a sufficient remaining level of the built-in battery 19 can be ensured in the ultrasound examination. In addition, since the built-in battery 19 is charged at a charging level lower than the first charging level in the on state of the power supply of the ultrasound probe 1, the adverse effect of the temperature rise of the built-in battery 19 on the life of the built-in battery 19 can be reduced.

It should be noted that, in the above description, the built-in battery 19 of the ultrasound probe 1 is charged by wireless charging, but the built-in battery 19 can also be charged by wired charging. A charging method of the built-in battery 19 is not particularly limited.

In addition, in the above description, the ultrasound probe 1 includes the image generation unit 13, but the ultrasound apparatus main body 3 can also include the image generation unit 13.

### Explanation of References

1: ultrasound probe

3: ultrasound apparatus main body

5: holding stand

11: transducer array

12: transmission and reception circuit

13: image generation unit

14: wireless communication circuit

15: ultrasound transmission and reception controller

16: communication controller

17: charging controller

18: probe controller

19: built-in battery

20: power receive coil

21: power switch

22: image acquisition unit

23: probe processor

31: wireless communication circuit

32: display controller

33: monitor

34: communication controller

35: main body controller

36: input device

37: built-in battery

38: main body processor

41: pulser

42: amplification section

43: AD conversion section

44: beam former

45: signal processing section

46: DSC

47: image processing section

What is claimed is:

1. An ultrasound probe that is wirelessly connected to an ultrasound apparatus main body, the ultrasound probe comprising:
a transducer array;
a built-in battery; and
a processor configured to:
control an on state and an off state of a power supply of the ultrasound probe; and
charge the built-in battery by using power supplied from an outside of the ultrasound probe,
wherein the processor is configured to charge the built-in battery at charging levels different depending on the on state and the off state of the power supply.

2. The ultrasound probe according to claim 1,
wherein the processor is configured to:
charge the built-in battery at a predetermined first charging level in the off state of the power supply; and
charge the built-in battery at a charging level lower than the first charging level in the on state of the power supply.

3. The ultrasound probe according to claim 2,
wherein the processor is configured to:
acquire an ultrasound image by transmitting and receiving ultrasound waves using the transducer array; and
wirelessly transmit the ultrasound image to the ultrasound apparatus main body, and
the processor is configured to charge the built-in battery at charging levels different depending on whether the ultrasound probe is in a connection state of being wirelessly connected to the ultrasound apparatus main bod or in a non-connection state of being not wirelessly connected to the ultrasound apparatus main body.

4. The ultrasound probe according to claim 3,
wherein the processor is configured to charge the built-in battery at a predetermined second charging level in the non-connection state, and charges the built-in battery at a third charging level lower than the second charging level in the connection state.

5. The ultrasound probe according to claim 1, further comprising:
a power receive coil,
wherein the processor is configured to charge the built-in battery by using the power wirelessly supplied from the outside of the ultrasound probe via the power receive coil.

6. The ultrasound probe according to claim 2, further comprising:
a power receive coil,
wherein the processor is configured to charge the built-in battery by using the power wirelessly supplied from the outside of the ultrasound probe via the power receive coil.

7. The ultrasound probe according to claim 3, further comprising:
a power receive coil,
wherein the processor is configured to charge the built-in battery by using the power wirelessly supplied from the outside of the ultrasound probe via the power receive coil.

8. The ultrasound probe according to claim 4, further comprising:
a power receive coil,
wherein the processor is configured to charge the built-in battery by using the power wirelessly supplied from the outside of the ultrasound probe via the power receive coil.

9. An ultrasound system comprising:
the ultrasound probe according to claim 1; and
the ultrasound apparatus main body that is wirelessly connected to the ultrasound probe.

10. A control method of an ultrasound probe including a transducer array, a built-in battery, a power supply controller that controls an on state and an off state of a power supply of the ultrasound probe, and a charging controller that charges the built-in battery by using power supplied from an outside of the ultrasound probe, the control method comprising:
charging the built-in battery at charging levels different depending on the on state and the off state of the power supply.
